(19) United States
(12) Patent Application Publication
(10) Pub. No.: US 2003/0003095 A1
(43) Pub. Date: Jan. 2, 2003

(54) Activated carbon for preventing pregnancy and sexually transmitted disease

(75) Inventors: Richard Joseph Sunberg, Oxford, OH (US); Sheri Anne Hunt, West Chester, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH (US)

(21) Appl. No.: 10/064,710

(22) Filed: Aug. 8, 2002

(51) Int. Cl. A01N 33/12; A61K 31/704; A61K 31/715; A61K 31/655; A61K 31/14

(52) U.S. Cl. 424/125; 514/54; 514/33; 514/643; 514/151; 514/723

(57) ABSTRACT

Activated carbon, as well as compositions and kits comprising the same, are effective for preventing pregnancy and sexually transmitted diseases including HIV.

### BACKGROUND OF INVENTION

The World Health Organization (“WHO”) announced that world population growth is the highest priority for global health. WHO also emphasized that sexually transmitted diseases (“STDs”) are of paramount concern. To this end, most of the global population growth is attributed to developing countries thereby straining already sparse resources. Many devices attempt to address the world's population growth and occurrence of STDs. Examples of such devices are described in: U.S. Pat. Nos. 5,756,681; 5,985,275; 5,952,009; and 6,165,493.

STD caused by C. trachomatis and N. gonorrhoeae account for 88 and 62 million new infections per year worldwide, respectively. These diseases may be asymptomatic, especially in females, yet lead to serious illnesses including pelvic inflammatory disease. While treatable with inexpensive antibiotics such as tetracycline and penicillin, the global cost of these two STDs alone is said to be approximately 2.5 billion U.S. dollars per year. Both of these infections may recur repeatedly, even in heavily exposed individuals such as sex workers, as antigenic variation of these STD agents permits evasion of host immunity.

Both of these STDs can be prevented with the use of barrier methods, i.e., male condoms. Use of a diaphragm with spermicidal nonoxynol-9 offers some protection. Both of these methods require compliance and professional counsel for correct use and benefit.

However, many problems still exist in condom and diaphragm use including availability, irritation, limited window of application time, and expense. There is a need for a safe, non-hormonal effective, women controlled, easy to use, affordable global contraceptive, that also will afford inexpensive, broad range efficacy against many STDs, including but not limited to Neisseria Gonorrhoeae, Chlamydia trachomatis and Human Immunodeficiency Virus (HIV).

For the foregoing reasons, there is a continuing need to find more effective ways to prevent pregnancy and STDs at a reduced cost. The present invention attempts to solve these problems.

### SUMMARY OF INVENTION

The present invention is based on the surprising discovery that many forms of activated carbon adsorb sperm and STD causative agents, thereby providing a safe and effective as well as inexpensive way to prevent a pregnancy or a STD.

The present invention provides for a method of preventing a pregnancy by administering a safe and effective amount of an activated carbon to a subject in need thereof.

The present invention further provides for a method of preventing a STD by administering a safe and effective amount of an activated carbon to a subject in need thereof.

The present invention further provides for a composition comprising (a) a safe and effective amount of an activated carbon; and (b) a safe and effective amount of a spermicidal agent, an anti-sexually transmitted disease agent, or mixtures thereof.

The present invention further provides for a composition comprising an inorganic oxide or carbonate substrate coated with an activated carbon.

The present invention further provides for a kit for preventing a pregnancy or preventing a STD comprising: (a) a unit dose form of an activated carbon; (b) a unit dose form of a spermicidal agent, an anti-sexually transmitted disease agent, or mixtures thereof; (c) a package containing components (a) and (b); and (d) optionally, directions for use thereof.

The present invention further provides for a kit for preventing a pregnancy or preventing a STD comprising: (a) an AC in a unit dose form; (b) a barrier agent; (c) a package containing components (a) and (b); and (d) optionally, instructions to the kit.

Lastly, the present invention provides for the use of activated carbon in the manufacture of a medicament for preventing a pregnancy or preventing a sexually transmitted disease in a subject in need thereof.

All documents cited are, in relevant part, incorporated herein by reference; the citation of any document is not to be construed as an admission that it is prior art with respect to the present invention.

### DETAILED DESCRIPTION

I. Method of Preventing a Pregnancy or STD

One aspect of the invention provides for a method of preventing a pregnancy by administering a safe and effective amount of an activated carbon to a subject in need thereof.

Another aspect of the invention provides for a method of preventing a STD by administering a safe and effective amount of an activated carbon to a subject in need thereof.

As used herein, “activated carbon,” is intended in the broadest sense. Generally, and without limitation, activated carbon (hereinafter “AC”) is a microcrystalline, nongraphitic form of carbon that is processed to develop internal porosity. AC is also known, to those skilled in the art and within the purview of the present invention, as activated charcoal or active carbon. The term “activated,” as used in AC, means a material that is carbonized and is subjected to an activation process. In turn, the activation process refers to the controlled pyrolysis of a carbon-containing starting material. Controlled pyrolysis is generally achieved by the oxidizing action of gases, such as steam or carbon dioxide, at high temperatures. Activation enhances the adsorptive power of carbon by developing fine pores in the substance. In turn, this porosity yields enhanced surface area that provides additional absorptive properties of AC. The size of the pores generally dictate the binding energy of the surface.

Many sources of material are contemplated to produce AC. Without limitation, sources of material include almost any carbonaceous material such as bone, hard and soft woods, rice hulls, nutshells, refinery residuals, peat, lignin, coal, coal tars, pitches, blood, cotton fibers, corncobs, fruit pits, and seedpod shells (like coconut). Many methods of activation are contemplated. Without limitation, suitable sources of material and methods of activation are described in Roy, Glenn M., “Activated Carbon Application in the Food and Pharmaceutical Industries, “Technomic Publishing Company, Inc, Lancaster, Pa. (1995) and the references cited therein. AC is typically available from commercial suppliers.

ACs of the present invention can be provided in a variety of shapes and sizes. For example, AC can be provided in simple forms such as granules, fibers, and beads. Furthermore, AC can be provided in the shape of a sphere, polyhedron, cylinder, as well as other symmetrical, asymmetrical, and irregular shapes. Still further, AC can also be formed into complex forms such as webs, screens, meshes, non-wovens, wovens, and bonded blocks, which may or may not be formed from the simple forms described above.

Non-limiting examples of AC include bone black, available from Sigma Chemical Corporation; United States Pharmacopeia (USP) quality AC, also available from Sigma Chemical Corporation; and AC cloth, available from Calgon Carbon Corporation.

As used herein, “prevent pregnancy,” means diminishing the risk of pregnancy in a mammalian female subject, preferably a human female. It is understood that “prevent” does not require the risk of a female subject becoming pregnant after sexual contact be completely thwarted.

As used herein, “prevent STD,” means diminishing the risk of a STD occurring in a mammalian subject, preferably humans. It is understood that “prevent” does not require the risk of a subject acquiring a STD after sexual contact be completely thwarted.

As used herein, “STD” means those diseases that are transmitted by STD pathogen through sexual contact. Non-limiting examples of these STD pathogens include, but are not limited to, bacteria, viruses, yeast and other fungal spores, helminthes, and protozoa. STDs include those selected from, but not limited to, the group consisting of Chlamydia, gonorrhea syphilis, genital herpes, acquired immunodeficiency syndrome (AIDS) and hepatitis.

The aspect of the invention that is based upon the method of AC preventing pregnancy stems from the surprising observation that sperm have a strong affinity for AC. It is observed under a light microscopy that sperm congregate around AC when mixed in aqueous media. Shortly thereafter, the sperm ultimately cease to be visible. Without wishing to be bond by theory, it is believed that AC, with its strong absorptive capacity, binds sperm with sufficient force to rupture the cell membrane thereby bursting the sperm, which leads to the sperm to become undetectable under light microscope.

Another mechanism, by which AC is believed to prevent pregnancy, without wishing to be bound by theory, is by adsorbing capacitating agents. As used herein, “capacitating agents” are those agents in vaginal fluid that provide the necessary biochemical cues to sperm to become viable upon ejaculation.

In another aspect of the invention, AC is bound (by absorption or otherwise) to a chemical or thermal means to attract sperm by mimicking biochemical attractant cues associated with an egg. Such chemical or thermal means would act to attract sperm or STD agents to AC thereby enhancing the anti-pregnancy or anti-STD effects of AC. Chemical means include, but are not limited to, those cumulus components that surround an egg. Thermal means include, but are not limited to, those chemicals that produce heat upon hydration.

As to preventing STDs, AC is known to bind pathogens. However, it is surprisingly observed that AC binds to Neisseria gonorrhoeae and Chlamydia trachomatis. As such, AC may be used to prevent a STD. Without wishing to be bond by theory, it is believed that the AC has the advantage of selectively binding to STD pathogens as compared to natural flora. Vaginally, natural flora are typically established in great numbers and often in close association with vaginal walls and thus not easily disrupted by the presence of AC. In contrast, STD pathogens are typically in “plankton form” (i.e., free-floating) and much fewer in numbers. Thus, STD pathogens will likely be more attracted to AC resulting in a greater impact on their overall population and thus decreasing their virulence. In view of the foregoing, AC may provide an advantage over other medicaments that are non-selective. For example, an antibiotic may non-selectively destroy the populations of a bacterial STD pathogen as well as natural flora of the vagina thereby subjecting the female subject to opportunistic infections such as a vaginal yeast infection. One skilled in the art will appreciate that STD pathogens need not be eliminated to prevent a STD in a subject.

AC may also provide additional advantages over other contraceptive or STD medicaments. For example, the mechanistic actions of sexual contact my increase the overall effectiveness of AC. Without wishing to be bond by theory, if AC is administered vaginally, for example, as a finely divided particulate before intercourse, the mechanistic action of intercourse will further disperse AC thereby likely increasing the overall effectiveness of AC. AC also provides the additional advantage of being expelled slowly from the vagina via natural expulsion. This provides for a longer effectiveness period between the administration of AC and sexual contact to increase the likelihood of compliance and to provide enhanced protection from multiple sexual contact events.

II. Composition and Kits

One aspect of the invention provides for compositions comprising (a) a safe and effective amount of an AC; (b) a safe and effective amount of an anti-STD agent, a spermicidal agent, or a mixture thereof; and (c) optionally, a pharmaceutically acceptable carrier.

Another aspect of the invention provides for composition comprising an inorganic oxide or carbonate substrate coated with AC.

Yet another aspect of the invention provides for kits comprising: (a) an AC in a unit dose form; (b) an anti-STD agent, a spermicidal agent, or a mixture thereof, in a unit dose form; (c) a package containing components (a) and (b); and (d) optionally, instructions to the kit.

Still yet another aspect of the invention provides for kits comprising: (a) an AC in a unit dose form; (b) a barrier agent; (c) a package containing components (a) and (b); and (d) optionally, instructions to the kit.

As used herein, “spermicidal agent,” is used in the broadest sense and means a non-AC, non-barrier agent that reduces: (1) the number of viable sperm; (2) sperm motility; or (3) sperm forward progression in the vagina. A non-limiting example of a spermicidal agent is nonoxynol-9.

As used herein, an “anti-STD agent,” is used in the broadest sense and means any non-AC, non-barrier agent that mitigates or interferes with an STD infection. In one embodiment, the different anti-STD agents that are within the scope of the invention are selected from the group consisting of a surfactant agent, anti-metabolite agent, competitive binding inhibitor agent and mixtures thereof. In one embodiment, the ant-STD agent is a surfactant agent. Non-limiting examples of a surfactant agent include saponin, nonoxynol-9, octoxynol-9, chlorhexidine, benzalkonium halide, triclosan and mixtures thereof. In one embodiment, the surfactant agent is nonoxynol-9. A non-limiting example of an anti-metabolite agent is azidothymidine (AZT™). Non-limiting examples of a competitive binding inhibitor agent include dextrans and carrageen-type polysaccharides. In one embodiment, the competitive binding inhibitor agent is dextran sulfate (molecular weight range from about 5,000 to about 10,000 kD). One skilled in the art appreciates that some anti-STD agents are spermicidal agents and vice versa.

As used herein, a “barrier agent,” is used in the broadest sense and means a device well known in the art that provides a physical barrier for the prevention of a pregnancy or STD. Non-limiting examples of a barrier agent include a condom or diaphragm.

The ACs, compositions, or kits that comprise the present invention may be administered topically to a mucosal surface of a mammalian subject, preferably human subject, more preferably a female subject, in a body cavity where sexual contact may occur, that includes the vagina, rectum and mouth, in the prevention of a pregnancy or STD. The present invention is typically formulated for the particular mode of administration, i.e., vaginally, rectally, or orally.

As used herein, a “safe and effective amount” of an AC, composition, or kit of the present invention is an amount that is effective to prevent a pregnancy or STD in a mammalian subject, preferably a human subject, more preferably a female human subject, without undue adverse effects (such as toxicity, irritation, or allergic response), commensurate with a reasonable benefit/risk ratio when used in the manner of the present invention. The specific “safe and effective amount” will, obviously, vary with such factors including, but not limited to, time between administration and sexual contact, multiplicity of sexual contacts, multiplicity of sexual partners, body cavity, risk of exposure to pathogens, the physical condition and reproductive age of the subject, the specific dose form to be used, the carrier employed, and the dosage regimen.

As previously described, one aspect of the invention provides for kits that comprise an AC and an anti-STD agent, spermicidal agent, or mixture thereof, in a unit dose form. As used herein, a “unit dosage form” is an amount of AC or agent that is suitable for administration to a mammalian subject, preferably a human subject, more preferably a human female subject, according to good medical practice. The unit dose forms preferably contain from about 1 milligram (mg) to about 1000 mg of the AC or agent. In one embodiment, the unit dose forms contain from about 3 mg to about 250 mg of AC or agent. If the AC and the spermicidal agent and/or anti-STD agent are combined in a single unit dose form, the unit does form preferably contains from about 1 mg to about 1,500 mg of the AC and the agent(s) combined.

In one embodiment, the present invention provides a weight percentage ratio of an spermicidal agent and/or an anti-STD agent relative to AC from about 0.001% to about 500%, in another embodiment from about 0.01% to about 30% ratio, in yet another embodiment a ratio of about 1% to about 15%.

The ACs, compositions, or kits that comprise the present invention can be administered to a subject in need thereof from about 72 hours or more to moments before or after sexual contact. In one embodiment, the present invention may be administered 48 hours before sexual contact. In another embodiment, administration before sexual contact is selected from the group consisting 24 hours, 12 hours, 8 hours, 4 hours, 2 hours, 1 hour, 30 minutes, 5 minutes, and 1 minute. In yet another embodiment, administration of the present invention is 5 minutes after sexual contact. In still yet another embodiment, the administration of the present invention is concurrent with sexual contact.

If, in an embodiment of the invention, the kit comprises AC in a unit dose form and an anti-STD agent, spermicidal agent, or mixture thereof comprises another unit dose form, these unit dose forms need not be administered concurrently. That is, these unit dose forms may be administered separately anytime from about 72 hours or more to moments before or after sexual contact. However, in another embodiment, these unit dose forms are administered concurrently.

A non-limiting example of a composition of the present invention is AC and nonoxynol-9. To this end, nonoxynol-9, or any other suitable spermicidal agent and/or anti-STD agent, may be reversibly bond to AC by depositing the nonoxynol-9 by filtration and subsequent solvent evaporation. For example, nonoxynol-9 may be added to a suitable solvent with a low boiling point, such as ethyl alcohol, at room temperature, at a ratio of about 0.1 to about 500 weight percentage ratio relative to AC. Thereafter, the solvent is evaporated at standard pressure or under vacuum to yield a composition of the present invention.

One skilled in the art will readily identify a suitable solvent to reversibly bind an anti-STD agent, spermicidal agent, or mixture thereof to the AC determined by the physicochemical nature of the agent. For example, ionic agent compatible solvents include water, methanol, ethanol, isopropanol, or in combination with each other. For example, in a hydrophilic system, co-solvents with water include polyethleneglycols, propyleneglycols, acetonitrile, methylene chloride, or in combination with each other. For example, in a hydrophobic system co-solvents with glycerin include alcohols, polyvinylpyrolidone, tetrahydrofuran, methyl isobutyl ketone, or in combinations with each other. Regarding non-AC agents, for example, nonoxynol-9 is a slightly water soluble; triclosan is water insoluble; and chlorhexidene digluconate is water-soluble.

Another non-limiting example of a composition of the present invention comprises an activated carbon coated particles (hereinafter “ACCP”) and a reversibly bond spermicidal agent, an anti-sexually transmitted disease agent, or mixtures thereof. ACCP is an inorganic oxide or carbonate substrate coated with AC. In one embodiment, the inorganic or carbonate substrate of ACCP is selected from the group consisting of silicon dioxide, titanium dioxide, zinc oxide, calcium oxide, calcium carbonate and mixtures thereof. In another embodiment, the carbonate substrate of ACCP is calcium carbonate. In yet another embodiment, the AC of ACCP is a thermoset phenolic resin. A suitable example of a thermoset phenolic resin is DURITE SC-409 B (1-5 weight percentage formaldehyde and 1-5 weight percentage phenol) from Borden Chemical Inc., Louisville, Ky. In still yet another embodiment the agent is nonoxynol-9. A composition comprising ACCP and a reversibly bond spermicidal agent, an anti-sexually transmitted disease agent, or mixtures thereof can be produced as previously described.

Generally, other active ingredients may also be included in the compositions and kits of the present invention. As used herein, “other active ingredients” for preventing a pregnancy or STD are non-AC, non-spermicidal agent, non-anti STD agent and non-barrier agent, ingredients selected from the group consisting of polyamides (e.g., powdered nylon-6), clays (e.g., zeolites, talc, diatomaceous earth, and the like.), pollens (e.g., pecan pollens), polysaccarides (e.g., chitosan), metal oxides (e.g., alumina) and mixtures thereof. Polymeric polyamides and inorganic oxides are available from BASF Corporation, Ledgewood, N.J. Chitosan is available from Vanson, Redmond, Wash. Zeolites are available from Degussa, Akron, Ohio. Lastly, clays are available from Southern Clay Products Inc., Gonzales, Tex.

The compositions and kits of the present invention may optionally contain a pharmaceutically acceptable carrier. The term “pharmaceutically-acceptable carrier,” as used herein, means one or more compatible solid or liquid filler diluents or encapsulating substances which are suitable for administration to a mammalian subject, preferably a human subject, more preferably a human female subject for the prevention of a pregnancy or a STD. The term “compatible,” as used herein, means that components of the composition are capable of being commingled with AC or the compositions and kits of the present invention, and with each other, in a manner such that there is no interaction that would substantially reduce the efficacy of the present invention under ordinary use situations. A pharmaceutically-acceptable carrier must, of course, be of sufficiently high purity and sufficiently low toxicity to render it suitable for administration to a mammalian subject. Non-limiting examples of a pharmaceutically-acceptable carrier include ointment, cream, gel, lotion, paste, jelly, spray or foam.

The choice of a pharmaceutically acceptable carrier to be used in conjunction with an embodiment of the present invention is basically determined by the way the embodiment is to be administered. For example, the compositions of the present invention can be applied in a body cavity, such the vagina, rectum, or mouth, or can be utilized in conjunction with a contraceptive device by applying the composition on a contraceptive device prior to sexual intercourse.

An AC or a composition of the present invention suitable for vaginal or rectal administration may be presented as pessaries, tampons, suppositories, creams, gels, pastes, jelly, foams or sprays or aqueous or oily suspensions, solutions or emulsions (liquid formulations), or films containing in addition to AC or composition, such carriers as are known in the art to be appropriate.

Liquid compositions according to the present invention would include an AC or a composition of the present invention in a pharmaceutically acceptable liquid carrier or diluent, such as purified water or a physiological saline solution. The liquid preparations may also contain conventional additives, such as suspending agents, emulsifying agents, non-aqueous vehicles (which may include edible oils) or preservatives.

Ointments, pastes, jellies, liquids, foams, gels and creams may, for example, be formulated with an aqueous or oil base with the addition of suitable thickening and/or gelling agents. Lotions may be formulated with an aqueous base or an oil base and will in general also contain one or more emulsifying agents, coloring agents, stabilizing agents, suspending agents, thickening agents or surfactants, such as a nonionic surfactant, for example, a polyoxyethylene higher alcohol ether or polyethylene glycol.

A gel preparation having a high viscosity can be prepared by adding a conventional thickening agent into the above-described liquid preparation. Non-limiting examples of thickening agents include cellulose lower alcohol ether, polyvinyl alcohol (“PVA”), polyvinylpyrrolidone (“PVP”) and polyoxyethylene oxypropylene glycol block copolymer (such as “PLURONIC”).

A film preparation for use in the present invention may be prepared by mixing an AC or composition of the present invention with the above discussed liquid preparation, with a film base, for example, hydroxypropylmethyl cellulose, chitosan, pullulan, glucomannan or polyacrylate ester.

A tampon-shaped preparation for use in the present invention may be prepared by coating a tampon-shaped core made of silicone resin with a polymer film containing an AC or composition of the present invention. Vaginal administration, wherein the carrier is a solid, such as a wax, can be in the form of unit dose suppositories (contraceptive membrane suppositories). Suitable carriers include cocoa butter, agarose, dextran or glycerogelatin, and other materials commonly used in the art, and the suppositories may be conveniently formed by admixture of ACs or compositions of the present invention with the softened or melted carrier(s) followed by chilling and shaping in molds. Vaginal rings containing inert materials can also be used to introduce an AC or composition into the vagina.

A vaginal preparation of the present invention should preferably have a pH value close to that of the vagina, i.e., 3 to 7, preferably 4 to 6. Similarly, a rectal or oral preparation should have pH value to that that of rectum and mouth, respectively. The pH may be adjusted by an acid or base which is nontoxic and nonirritating to humans, for example, an organic acid such as acetic acid, or citric acid, or a weak base, such a sodium hydrogen carbonate or sodium acetate.

Additives that may be used in accordance with the present invention may include excipients (such as starch, dextrin, mannitol, cyclodextrin and traganth) binding agents, fillers, colorants (such as beta-carotin) lubricants, isotonic agents (such as sodium chloride or glucose) disintegrants, antioxidants (such as ascorbic acid, erythorbic acid, or a salt or ester thereof) or wetting agents. With respect to additives, which are utilized in pharmaceutical compositions, see Remington's Pharmaceutical Sciences, (latest edition), edited by Gennaro, A. R., Mack Publishing Company, Easton, Pa.

There are many advantages in using the compositions and kits of the present invention. The use of a composition or kit offers several advantages over the use of the anti-STD agent or spermicidal alone. Not only does AC prevent a pregnancy or STD in its own right, but enhances the pregnancy preventive effect of the spermicidal or anti-STD effect of the anti-STD agent by: (1) prolonging contact with the host body cavity tissue; or (2) prolonging contact with sperm or STD pathogen, respectively. The effective use of an anti-STD or spermicidal agent at lower concentrations enables a lower amount of the agent to be used per dose. A lower amount of these agents will likely decrease irritation to user or sexual partners. The decrease in the irritation to body cavity mucosal surfaces or to other epithelial tissue encourages more faithful use of the composition or kit thereby resulting in a more effective prevention of a pregnancy or STD. Compliance is also enhanced given the ability for the user to administer the invention up to, if not more, about 72-hours before sexual contact. Costs for the effective prevention of a pregnancy and STD are reduced, since a lower amount of these agents are needed.

The present invention may also increase the effectiveness of the use of Nonoxynol-9 by decreasing the irritation to the mucosal linings of the vagina and rectum. Recent studies have suggested that the use of Nonoxynol-9, when used exclusively with a condom during sexual intercourse, irritates these mucosal linings. This irritation is believed to increase the probability of contracting STDs. Thus, utilizing AC, in a composition or kit of the present invention, may increase the effectiveness of Nonoxynol-9 when used in conjunction with a barrier agent.

Except as otherwise noted, the articles “a”, “an”, and “the” mean “one or more”.

Except as otherwise noted, all ratios are weight ratios.

### EXAMPLE 1

AC and compositions comprising AC are evaluated in vitro for the prevention of bacterial STD. Stock strains and/or clinical isolates of Neisseria gonorrhoeae are selected. One to two colonies on Thayer-Martin media are emulsified in 9 mL of nutrient broth (tryptic soy broth with 5% Fildes enrichment) prewarmed to 35° C. to create a positive stock. Each AC sample material (50 mg unless otherwise noted) to be tested is pre-wet with 0.5 mL of sterile nutrient broth, prewarmed to 35° C., and briefly vortexed. Then, 0.5 mL of positive stock is added, mixed briefly and incubated in an oscillating water bath at 35° C. (+2° C.) for 60-75 minutes. Following incubation, the liquid contents of the test tube are pipetted into an empty 3 cc syringe barrel, attached to a Gelman 5-micron Acrodisc syringe filter. The barrel of the syringe is inserted, and the filtered liquid dispensed into a clean test tube. A 10 μL quantitative loop is used for semiquantitative plating of the filtered liquid. Colony counts are reported as a log of 10 per mL, with the limits as 10 per mL to >10⁶ per mL. If needed, organism density of the nutrient broth is adjusted up or down to reveal differences between pre- and post-treatment, if such differences exist.

Positive controls and patient positive material from genital isolates of Chlamydia species (presumed by their isolation site to be C. trachomatis) are randomly selected; this positive stock is consisted of supernatant from positive wells, subjected to one freeze-thaw cycle. AC sample materials (50 mg unless otherwise noted) are prewet with 0.4 mL of Bartel's transport media and briefly vortexed. Then, 400 μL of media from the positive stock material are added to each tube, briefly vortexed, and incubated at 35° C. (+2° C.) for 60-75 minutes in an oscillating water bath. Following incubation, the liquid contents of the test tube are pipetted into an empty 3 cc syringe barrel, attached to a Gelman 5-micron Acrodisc syringe filter. The barrel of the syringe is inserted, and the filtered liquid dispensed into a clean test tube. Then, 0.5 mL of this filtered liquid is pipetted into the well of a commercial (Bartel's) microtiter plate containing McCoy cells for Chlamydia detection. All Chlamydia culture wells are treated as a routine Chlamydia culture. Briefly, these steps consist of one hour of centrifugation at 2000×g, incubation for 48 hours at 36° C. in 5% CO₂, aspiration of the overlay media, ethanol fixation, and immunofluorescence microscopy. The untreated control is handled the same as the treated samples, i.e., it is incubated and filtered.

### EXAMPLE 2

AC and compositions comprising AC are evaluated for the prevention of pregnancy. A sample of human sperm is diluted with Gibco BRL F-10 Nutrient Mixture (Ham) with L-glutamine solution so that the final sperm concentration is 12.5 million sperm per milliliter. The sperm viability (alive) is about 50% thereby indicating a normal range. The activated carbons to be tested is placed into 15 mL plastic test tubes to which 500 microliters of the diluted sperm solution was added to each tube. The tubes are gently mixed and the solids are allowed to settle for no more than 5 minutes. The relatively clear layer supernatant is sampled by and applied to a Millennium Sciences Inc. Cell-VU Disposable Counting Chambers sperm counting grid chamber for a microscopic manual count of total sperm, viable sperm and dead sperm. The data is normalized to the control with the control sperm concentration at 12.5 MM sperm/mL.

### EXAMPLE 3

In another example, AC and compositions comprising AC are evaluated for the prevention of pregnancy in artificially inseminated (Al) New Zealand White Rabbits under two study conditions. All control rabbits in both studies are inseminated, with sperm delivered in a dilution with Gibco F-10 Nutrient Mixture (Ham) with I-glutamine, total volume 0.50 mL.

Study I: In this study the treatment group is inseminated with the same sperm dilution and volume as are the control rabbits except that 50 mg of AC or a composition comprising AC is added to the sperm dilution and mixed by gentle inversion just prior to insemination.

Study II: In this study the AC or a composition comprising AC is introduced into the rabbit vagina several minutes before the rabbits are inseminated.

Procedure: Fresh semen is collected from bucks and pooled. This semen sample is examined for sperm count and diluted to Al specifications. Final dilution is from about 4 mL of semen to about 8 mL of semen final volume. The diluent is GibcoBRL F-10 Nutrient Mixture (Ham), Cat. # 11550-035. An evaluation of this sperm dilution indicates that greater than 50 million sperm per mL is present with about 60% viability and that a 0.50 mL insemination volume would contain sufficient numbers of viable sperm to expect pregnancies based on literature values.

All does received an ear vein hormone injection to induce ovulation just prior to insemination.

Results are determined by birth observations after a 30 to 32 day gestation period.

### EXAMPLE 4

An ACCP is made by utilizing silicon dioxide as the inorganic oxide substrate and Durite SC-409 B from Borden Chemical Inc. as the AC carbon source. 1000 grams of silica are mixed thoroughly with 1400 milliliters of deionized water using a 20,000 r.p.m, rotor sator high shear mixer (Janke & Kunkely, Tekmar Co., Model No. SD45) for 15 minutes. Mixing efficiency is determined by particle size distribution relative to starting material with 80 percent match for particle-size distribution as acceptable. Thereafter, 30 grams of DURITE SC 409B is added and mixed for 5 minutes at 20,000 r.p.m. to yield a resin coated inorganic oxide.

The resin inorganic oxide is spray dried using a portable Nitro Type HT Spray dryer under the following conditions: inlet temperature of 250° C.; outlet temperature of 100° C.; air flow of 40 millimeters water pressure drop across orifice plate; liquid flow of 5 kilograms per hour; a rotary atomizer nozzle; atomizing air of 5 bar to spin atomizer; yielding product at 2.5 kilograms per hours; under a 150 mmWs vacuum; with an average particle size of 25 micron; and an average water content of 7 weight percent.

The sprayed resin is placed in a box furnace (VWR, Lindberg Blue 1500° C. with programmable temperature control). The nitrogen flow is at 90 mm+/−10 mm with the temperate increased from 25° C. to 650° C. in four hours and held at 650° C. for two additional hours. Thereafter, the furnace is cooled to below 100° C. before the furnace is opened.

Particle size is analyzed utilizing a Horiba Laser Scattering Particle Size Distribution Analyzer LA-910 under standard conditions.

### EXAMPLE 5

A composition comprising AC and 40% releaseably bound Nonoxynol-9 is provided. Specifically, CA 191 (Norit USP CAS 7440-44-0) and TERGITOL NP-10 (nonionic)(J T Baker, CAS 26027-38-3) are provided as the AC and Nonoxynol-9 sources, respectively. 10.0 g (3.2%) Activated Carbon CA 191 (Norit USP CAS 7440-44-0) a black solid, is slurried with 100.0 mL (32.2%) deionized water. 56.0 mL (18%) Tergitol™ NP-10 (nonionic) [CAS 26027-38 3] [J T Baker] a yellowish liquid, is added with stirring in fume hood at room temperature. 144.1 mL (46.4%) of punctilious ethanol, clear liquid, is added with stirring. 5 hours post mixing the dispersion is filtered using a large plastic funnel with a Whatman # 54, 24 cm filtration paper. Sample is dispersed on filter paper and is allowed to air dry for 24 hours at room temperature prior to packing into sample vials. Thermogravimetric analysis yields AC reversibly-bond with 40% Tergitol™ NP-10.

### EXAMPLE 6

A composition comprising AC and 15% releaseably bond Nonoxynol-9 is provided. Specifically, CA 191 (Norit USP CAS 7440-44-0) and TERGITOL NP-10 (nonionic)(J T Baker, CAS 26027-38-3) are provided as the AC and Nonoxynol-9 source, respectively. 5.0 g (3.2%) Activated Carbon CA 191 (Norit USP CAS 7440-44-0) a black solid, is slurried with 500.0 mL (32.2%) deionized water. 3.0 mL (1.9%) Tergitol™ NP-10 (nonionic) [CAS 26027-38 3] [J T Baker] a yellowish liquid, is added with stirring in fume hood at room temperature. 97.0 mL (62.6%) of punctilious ethanol, clear liquid, is added with stirring. 5 hours post mixing the dispersion is filtered using a large plastic funnel with a Whatman # 54, 24 cm filtration paper. Sample is dispersed on filter paper and is allowed to air dry for 24 hours at room temperature prior to packing into sample vials. Thermogravimetric analysis yields AC reversibly bond with 15% Tergitol™ NP-10.

### EXAMPLE 7

A composition comprising ACCP and triclosan is provided. 14.475 g triclosan is dissolved in 100 ml of a solvent consisting of 70% ethanol and 30% water. 20 cm³ of this triclosan solution is pipetted onto 1 g of zinc oxide based ACCP in a 100 ml beaker, which is then covered. The solution is stirred using a magnetic stirrer for 10 minutes and left to stand overnight. Next day, after a further stirring for 5 minutes, the solution is transferred to a filtration column fitted with a 20 μm filter. The solution is vacuum filtered at 15″ Hg using a 12-port vacuum manifold until the sample material became a dry powder. The sample material particles are then washed with more solvent to remove any free, un-bound triclosan. 10 cm³ of the ethanol: water solvent is pipetted onto the dry, loaded sample material in the filtration column and stirred with a spatula. The sample material slurry is again vacuum filtered at 15″ Hg until the sample material could be recovered as a dry powder of a composition of ACCP reversibly bond with triclosan.

### EXAMPLE 8

A composition comprising ACCP and chlorhexidene digluconate is provided. 10 cm³ of chlorhexidene digluconate solution (20% aqueous solution) is pipetted onto 1 g of silicon dioxide based ACCP in a 100 ml beaker and covered. The covered beaker is also wrapped in foil to prevent any photo degradation of chlorhexidene digluconate. The solution is stirred using a magnetic stirrer for 10 minutes and left to stand overnight. After a further stirring for 5 minutes the slurry is transferred to a filtration column fitted with a 20 μm filter. The solution is vacuum filtered at 15″ Hg using a 12-port vacuum manifold until the sample material became dry powders. The sample material particles are then washed with more water to remove any free, un-bound chlorhexidene digluconate. 10 cm³ of water is pipetted onto the dry, loaded sample material in the filtration column and stirred with a spatula. The sample material slurry is again vacuum filtered at 15″″ Hg until the loaded sample material could be recovered as a dry powder of a composition of ACCP reversibly bond with chlorhexidene digluconate.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A method of preventing a pregnancy by administering a safe and effective amount of activated carbon to a subject in need thereof.

2. A method of preventing a sexually transmitted disease by administering a safe and effective amount of an activated carbon to a subject in need thereof.

3. The method of claim 2 wherein the sexually transmitted disease is selected from the group consisting of chlamydia, gonorrhea, syphilis, genital herpes and acquired immunodeficiency syndrome.

4. A composition comprising:
(a) a safe and effective amount of an activated carbon; and
(b) a safe and effective amount of a spermicidal agent, an anti-sexually transmitted disease agent, or mixtures thereof.

5. The composition of claim 4, wherein the activated carbon is selected from the group consisting of activated carbon coated particles and activated cloth carbon.

6. The composition of claim 4, wherein the spermicidal agent is nonoxynol-9.

7. The composition of claim 4, wherein the anti-sexually transmitted disease agent is selected from the group consisting of surfactant agent, anti-metabolite agent, competitive binding inhibitor agent and mixtures thereof.

8. The composition of claim 7, wherein the surfactant agent is selected from the group consisting of saponin, nonoxynol-9, octoxynol-9, chlorhexidine, benzalkonium halide, triclosan and mixtures thereof.

9. The composition of claim 7, wherein the anti-metabolite agent is azidothymidine.

10. The composition of claim 4, further comprising other active ingredient for preventing a pregnancy or preventing a sexually transmitted disease, wherein the other active ingredient is selected from the group consisting of polyamides, clays, pollens, polysaccharides, metal oxides, metal carbonates and mixtures thereof.

11. A composition comprising an inorganic oxide or carbonate substrate coated with an activated carbon.

12. The composition of claim 11, wherein the inorganic oxide substrate is selected from the group consisting of silicon dioxide, titanium dioxide, zinc oxide, calcium oxide, calcium oxide and mixtures thereof.

13. The composition of claim 12, wherein the activated carbon is a safe and effective amount and a thermoset phenolic resin.

14. The composition of claim 11 further comprising a spermicidal agent, an anti-sexually transmitted disease agent, or mixtures thereof.

15. A method of preventing a pregnancy by administering a safe and effective amount of a composition comprising an inorganic oxide or carbonate substrate coated with activated carbon to a subject in need thereof.

16. A method of preventing a sexually transmitted disease by administering a safe and effect of amount of a composition comprising an inorganic oxide substrate coated with activated carbon to a subject in need thereof.

17. A kit for preventing for preventing a pregnancy or preventing a sexually transmitted disease comprising:
(a) a unit dose form of an activated carbon;
(b) a unit dose form of an spermicidal agent, an anti-sexually transmitted disease agent, or mixtures thereof;
(c) a package containing components (a) and (b); and
(d) optionally directions for use thereof.

18. A kit for preventing for preventing a pregnancy or preventing a sexually transmitted disease comprising:
(a) a unit dose form of an activated carbon;
(b) a barrier agent;
(c) a package containing components (a) and (b); and optionally directions for use thereof.
